(19) United States
(12) Patent Application Publication
(10) Pub. No.: US 2010/0036238 A1
(43) Pub. Date: Feb. 11, 2010

(54) DEVICE AND METHOD FOR ASSESSING EXTENSION OF A DEPLOYABLE OBJECT

(75) Inventors: Michael R. Neidert, Co. Galway (IE); Kenneth C. Gardeski, Plymouth, MN (US); Claude A. Vidal, Santa Barbara, CA (US); Russell J. Redmond, Goleta, CA (US); Laurent G. Verard, Andover, CO (US)

Correspondence Address: MEDTRONIC, INC., 710 MEDTRONIC PARKWAY NE, MINNEAPOLIS, MN, 55432-9924, US

(73) Assignee: Medtronic, Inc., Minneapolis, MN (US)

(21) Appl. No.: 12/483,692

(22) Filed: Jun. 12, 2009

Related U.S. Application Data
(60) Provisional application No. 61/061,441, filed on Jun. 13, 2008.
(60) Provisional application No. 61/196,707, filed on Mar. 9, 2009.

(51) Int. Cl. A61B 5/05

(52) U.S. Cl. 600/424

(57) ABSTRACT

A medical instrument assembly is disclosed. The medical instrument has proximal and distal ends. A receiver coil being mounted proximate to a distal end of the medical instrument, the receiver coil being electrically connectable at a proximal end of the medical instrument. A deployable object being disposed and movable within the medical instrument. The deployable object bearing a high magnetic permeability material located proximate to the receiver coil. The high magnetic permeability material and the receiver coil combining to form an inductive element having an inductance that varies in a predetermined manner with the position of the deployable device relative to the receiver coil. A determination of the inductance being performed at the proximal end of the delivery catheter indicates the extension of the deployable device through the medical instrument.

### CROSS-REFERENCE TO RELATED APPLICATION

This application claims the benefit of U.S. Provisional Application Nos. 61/061,441, filed on Jun. 13, 2008 and 61/196,707, filed on Jul. 31, 2008, which are hereby incorporated by reference in their entirety.

### TECHNICAL FIELD

The present disclosure relates to medical instruments and, more particularly, to medical instruments for inserting an object within the body of a patient.

### BACKGROUND

For many medical applications, a needle or other delivery device or deployable object is deployed using a medical instrument, such as a catheter based system. Such medical instruments often have deflectable tips to assist inserting them into position. As a result, a fundamental problem with such systems is determining the distance that the deployable object extends beyond the distal tip of the medical instrument. For example, compression of the tip of the medical instrument during deflection may cause the extension distance of the deployable object not to be the same as the distance set at the proximal end of the medical instrument. For example, for a deflectable catheter that comprises both a needle delivery system and a pull-wire, the pull-wire force results in compression of the softer segment of the distal catheter tip when the tip is deflected. After compression, the needle injection depth set at the proximal end of the catheter does not correspond to the actual needle injection depth at the distal end of the catheter. This can be particularly problematic when the tissue injection depth must be very precise, such as when used with thin tissues, where the injection depth must be accurate.

One method of controlling the extension of a needle delivered by a catheter employs a collar on the needle and a stop inside the lumen of the catheter at the distal tip. In such a system, the needle can only be advanced until the collar on the needle abuts against the stop inside the catheter lumen. While such a system prevents the needle from being overextended, it does not allow the operator to select a desired amount of extension but rather the operator is limited to the amount of extension provided by the relative positions of the collar and the stop.

### BRIEF DESCRIPTION OF THE DRAWINGS

Aspects and features of the present invention will be appreciated as the same becomes better understood by reference to the following detailed description of the embodiments of the invention when considered in connection with the accompanying drawings, wherein:

FIGS. 1A-1B are schematic diagrams that depict needle extension in a prior art catheter;

FIG. 2 is a cross-section of an exemplary catheter containing a deployable object;

FIG. 3 is a needle with high magnetic permeability cores;

FIG. 4 is a graph of induction versus needle extension;

FIGS. 5a-5c are cross-sections of a deployable object within a catheter according to embodiments of the invention; and

FIG. 6 is a flow diagram depicting a method of using a medical instrument with a deployable object.

### DETAILED DESCRIPTION

The following detailed description is exemplary in nature and is not intended to limit the scope, applicability, or configuration of the invention in any way. Rather, the following description provides practical illustrations for implementing exemplary embodiments of the present invention.

Embodiments of the present disclosure are useful with medical instruments for inserting deployable objects within the body of a patient. After placing the medical instrument into a patient's body, a deployable object, which is disposed and moveable within the medical instrument, is extended from the medical instrument and into the patient's body. Such medical instruments include catheters, such as rigid and flexible catheters, endoscopes such as neuroendoscopes, bronchoscopes, chronic total occlusion catheters and surgical robots, for example. The deployable objects which may be delivered through the medical instrument include, for example, needles and other delivery devices, guide wires such as for cardiac leads, chronic total occlusion crossing guidewires, biopsy tools, ablation probes, and sensors. An exemplary needle system which may be used is disclosed in U.S. patent application Publication Number 2007/0164900 and 2005/0277889, the relevant portions of which are hereby incorporated by reference. When the deployable object is a delivery device, it may be used to deliver therapeutic fluids including biological agents such as genetic vectors, cells, proteins or chemical agents such as drugs. The deployable object may be a needle for ablation such as radiofrequency ablation for local necrosis of tissue such as small tumors or for cardiac rhythm management. For such uses, accurate placement of the deployable object such as placement of a needle at a precise depth in the tissue is particularly important so that the delivered substances can be precisely placed in the desired locations and at the desired depth in the tissue. Therefore embodiments of the present disclosure allow for the use of medical instruments for accurate control over the delivery of substances into tissue.

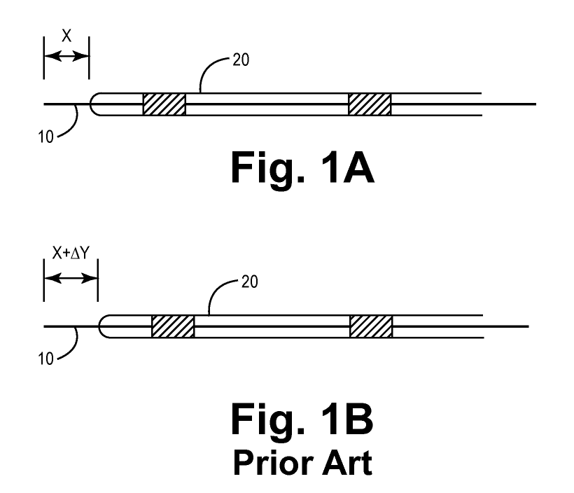

In catheters and other medical instruments having a deflectable tip, the deflectable tip is comprised of a material softer than the more proximal shaft. In such medical instruments, the difference in stiffness between the distal and more proximal portions of the instrument allows the tip to deflect more than the shaft, such as under the force of a pull-wire, resulting in an overall deflection of the tip. However, in addition to bending, the softer distal segment also compresses under the deflecting force. An example of this compression is shown in FIG. 1A-1B which depicts a prior art needle 10 being deployed from the tip of a catheter 20. When the deployment of the needle 10 is measured or determined at the proximal end of the catheter, it is measured as being x mm as shown in FIG. 1A. However, because of compression of the catheter tip, the actual distance of needle deployment is x+Δy, where Δy equals the amount of compression of the catheter 20, as shown in FIG. 1B. This compression occurs during the tip deflection because the pull wire is not situated on the neutral bending axis of the catheter shaft, resulting in combined bending and compression of the shaft. Therefore a measurement of needle deployment at the proximal end of the catheter underestimates the needle deployment by an amount equal to Δy. Furthermore, this compression is exacerbated when the medical instrument is placed within a patient's body. After a period of time within the body, the material of the medical instrument softens, resulting in an increased amount of compression, the amount of which is unpredictable.

Embodiments of the present disclosure comprise a system to measure the distance that an object is deployed from the distal tip of a medical instrument at the distal end of the instrument. Measuring the distance of extension of a deployable object at the distal tip of the medical instrument is more complicated than measuring extension at the proximal end because the distal tip is within the body of the patient, such as within the heart, at the time of deployment and therefore extension cannot be measured directly. Certain embodiments of the present disclosure therefore use an electromagnetic system and current induction to track and measure the extension of the deployable object beyond the distal tip of the medical instrument.

In some embodiments, the present disclosure is used for accurately injecting a needle into thin tissue a known distance. For example, the present disclosure may be used in thin tissues such as the right atrium, infarcted myocardium which has undergone remodeling, or vascular tissue. Such tissues may be injected with pacemaker cells, for example. In some applications, the tissue into which the material is injected may be only 3 or 4 mm, which is possible due to the accurate injection depth provided by embodiments of the present disclosure.

Certain embodiments of the present disclosure use an electromagnetic source and electromagnetic detectors or receivers for detecting the amount of extension of a deployable device. A generator is positioned external to the patient that sets up an oscillating magnetic field in the general area of the patient where the catheter will be deployed and acts as a transmitting source. The medical instrument or the deployable device includes an electromagnetic receiver, such as a receiver coil. In some embodiments, the transmitting source could be a coil, such as a coil which is about the size of the receiver coil, with the transmitting source in close proximity to the receiver coil, such as about 1 or 2 centimeters proximal to the receiver coil. The medical instrument is inserted into a patient's body, and the oscillating magnetic field induces a current in the receiver, within the body of the patient disposed within the magnetic field. Within the medical instrument and deployable object, the system uses high magnetic permeability cores, such as ferrous cores, which move relative to the receivers due to the motion of the deployable object. This motion results in a varying inductance in the receiver coil circuit. The electromagnetic field causes a current in the receiver coils, but this current is also effected by the high magnetic permeability cores, such as ferrous cores. For example, as the high magnetic permeability cores may begin at a position centered relative to the receiver coils, where the inductance of the combined cores and coils is highest. As deployable object is extended, the cores and coil are moved away from each other and the inductance (and therefore the current induced by the magnetic field) decreases. By monitoring the changes in the induced current or the inductance, the system can therefore determine the location of the high magnetic permeability cores relative to the receiver coils. This distance determination is then used to determine how far the deployable device has been moved away from the medical instrument.

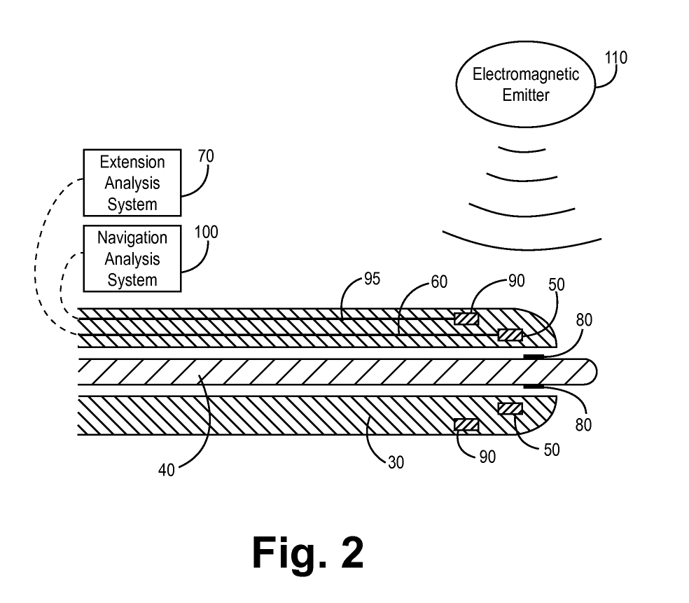

In one embodiment of the present disclosure, one or more high magnetic permeability cores are located on the deployable object and one or more receivers comprising receiver coils are located at or near the distal tip of the medical instrument. An example of this embodiment is shown in FIG. 2 which depicts a cross-section of a catheter 30 and deployable object 40. The receiver coil 50 is connected to a conductor 60 which extends through the catheter 30 to an extension analysis system 70. The distal tip in this example may be slightly compressed, as described above, during navigation. In some embodiments, the conductors 60 are a twisted pair of wires bonded to the receiver coil 50 and which run the length of the catheter and exit at the proximal end. The electromagnetic field causes a current to flow from the receiver coil 50 through the conductor 60, to be detected by the extension analysis system 70. As the deployable object advances, the high magnetic permeability core 80 on the deployable object 40 moves beneath the receiver coil 50. The high magnetic permeability core 80 induces an increase in current when it is moved in close proximity to the receiver coil 50. The increase in current is detected by the extension analysis system 70. The extension analysis system 70 interprets the change in current to determine the location of the high magnetic permeability core 80 relative to the extension receiver coil 50, which it then correlates to the amount of extension of the deployable object 40. In this way, the actual amount of extension of the deployable object 40 beyond the distal tip of the catheter 30 can be determined even in the presence of compression of the catheter tip. Embodiments of the present disclosure may include a single high magnetic permeability core 80 or more than one high magnetic permeability core 80. In some embodiments, the system includes three or more high magnetic permeability cores 80 located on the distal end of the deployable object 40. The high magnetic permeability cores 80 may be ferritic and may be comprised of pure iron, supermalloy or PERMALLOY™, for example. PERMALLOY™ is a nickel iron magnetic alloy. Generically, PERMALLOY™ refers to an alloy with about 20% iron (Fe) and 80% nickel (Ni) content. PERMALLOY™ has a high magnetic permeability, low coercivity, near zero magnetostriction, and significant anisotropic magnetoresistance. Supermalloy is an alloy composed of about Ni (79%), molybdenum (Mo) (5%), and Fe. Other percentages of elements can also be used for PERMALLOY™ and supermalloy.

In the embodiment shown in FIG. 2, the high magnetic permeability core is located on the deployable object 40 itself and is close to the tip of the deployable object 40. Alternatively, for certain deployable objects 40 such as needles or other metallic objects, at least a portion of the deployable object 40 itself, such as the distal tip, may be comprised of a ferritic material or material with high iron content such that the deployable object 40 itself functions as a high magnetic permeability core 80. For example, for embodiments in which the deployable object is a needle, the needle may be comprised of AISI 400 series stainless steel. Thus the high magnetic permeability core 80 does not necessarily have to be separate from the deployable object 40 but may be a part or component of the deployable object 40.

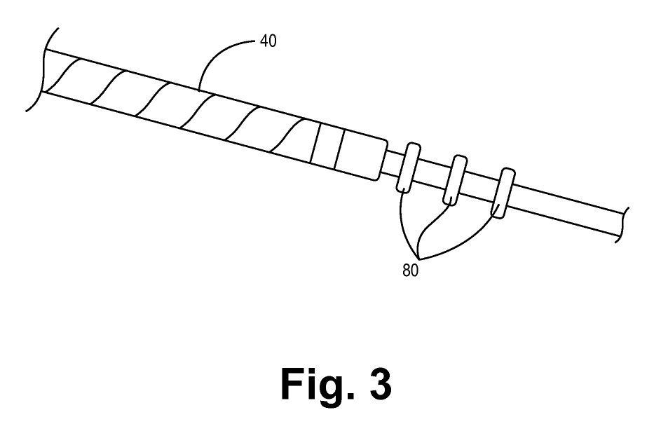

FIG. 3 depicts a deployable object 40 which is a needle for use with a catheter 30 according to embodiments of the present disclosure. As shown, the needle includes three high magnetic permeability cores 80 spaced apart at a location near the distal end of the needle. In this embodiment, the three cores 80 allow for a longer axial distance to be tracked than the use of one or two cores. For example, in some embodiments, the inductance gain due to a core 80 may be minimal beyond about 2 or 3 millimeters of distance between the core 80 and the coil 50. By using a deployable object 40 having multiple cores 50, the cores 50 may be spaced every two millimeters, for example, in order for the rising and falling inductance to be tracked as the deployable object 40 is advanced over a longer distance than would be possible using a single core 50.

High magnetic permeability cores 80 are comprised of high permeability magnetic material. This includes material which can be magnetized in response to a magnetic field. Such material may have a relative permeability of about 28,000 or more, for example. The high permeability magnetic core 80 may be comprised of one or more materials including cobalt, nickel, steel, iron, purified iron, silicon iron, mumetal, supermalloy, METGLAS®, AISI 400 Series stainless steel, or other similar material.

Embodiments of the present disclosure include one or more extension electromagnetic receiver coils 50 for assessing extension, also referred to herein as extension receiver coils 50. In the embodiment shown in FIG. 2, there is one electromagnetic receiver coil 50, located in the distal tip of the catheter 30. In some embodiments, such as the embodiment shown in FIG. 2, the electromagnetic receiver coil 50 comprises a coiled wire within the catheter 30 or other medical instrument which forms one or more loops around the lumen of the catheter 30 or other medical instrument. By looping around the lumen, the coiled wire occupies a minimum volume within the catheter or other medical instrument so that it maintains as thin a profile as possible while at the same time allowing for the formation of a loop having a large diameter. Examples of appropriate receiver coils 50 and associated components include the receiver assemblies disclosed in U.S. patent application Publication Number 2007/0164900, the relevant portions of which are hereby incorporated by reference.

One or more electromagnetic sources or transmitters emit a magnetic field into the space occupied by a patient undergoing catheterization. Examples of appropriate sources include the electromagnetic source used in the Medtronic StealthStation and the electromagnetic source disclosed in U.S. patent application Publication Number 2004/0097804, the relevant portions of which are hereby incorporated by reference.

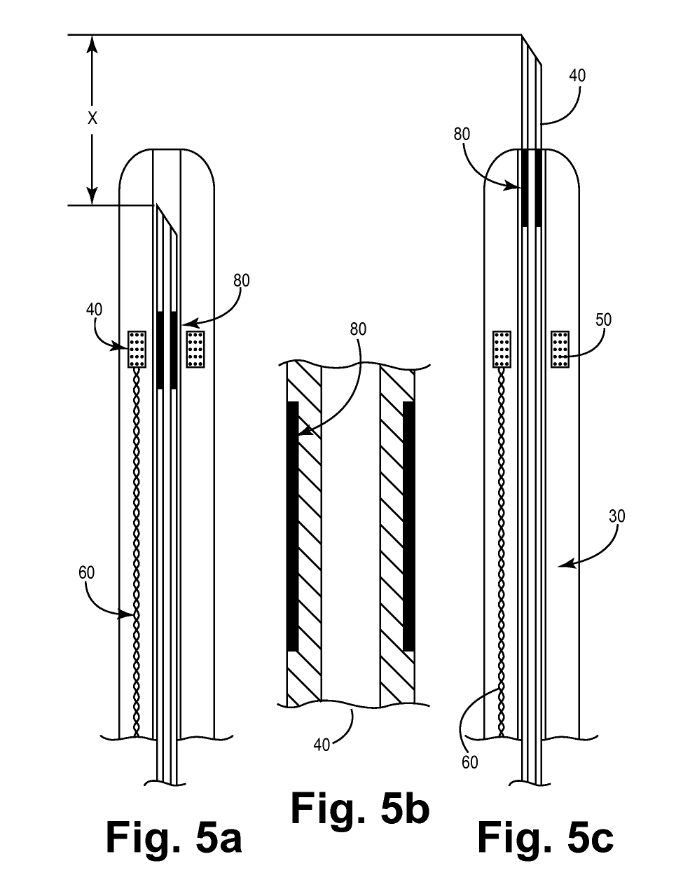

One embodiment of the present disclosure is shown in FIGS. 5a-5c. In FIG. 5a, the deployable object 40 including a high permeability core 80 is located within the catheter 30, with the high permeability core in proximity to the receiver coil 50. FIG. 5b shows a cross-section of the deployable object 40, with the high permeability core 80 within the wall of the deployable object 40. In FIG. 5c, the deployable object 40 is shown advanced by an amount x, such that the high permeability core 80 is spaced apart from the receiver coil 50. The movement of the high permeability core relative to the receiver coil results in a change in inductance which correlates to the distance x.

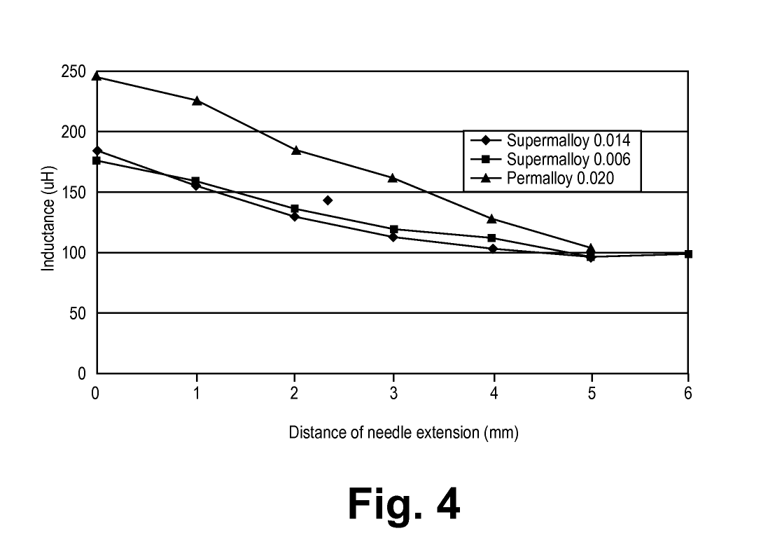

The combination of a receiver coil 50 with a moveable internal high permeability material creates a variable inductance that may be measured. If the high permeability material is mounted to or forms part of the deployable object 40, the inductance level may be used to determine the relative location of the receiver coil 50 and the deployable object 40. FIG. 4 provides a plot of inductance versus needle extension for an embodiment of the present disclosure. FIG. 4 demonstrates that the measured inductance as the needle, which has a high magnetic permeability core 80, extends through an extension electromagnetic receiver coil 50 in a catheter 30 tip. At a needle extension of zero, the inductance is at a maximum and the core 80 is centered within the extension receiver coil 50. As the needle and its high magnetic permeability core 80 is extended and moves away from the extension receiver coil 50, the inductance drops to the inductance of the coil. As shown in FIG. 4, the relationship between inductance and needle extension is approximately linear until inductance drops to a baseline level where it is no longer affected by the high magnetic permeability core 80. It should also be noted that, in some embodiments, the inductance may be measured while the high magnetic permeability core 80 is in motion. The measured induction correlates with the proximity of the high magnetic permeability core 80 to the extension electromagnetic receiver coil 50. Because of this direct relationship between inductance and needle extension, embodiments of the present disclosure are able to determine the precise amount of needle extension by measuring inductance. The system correlates the measured induction to the distance of extension of the deployable object, such as through the use of a calibration curve, which may be similar to the curves shown in FIG. 4. FIG. 4 also demonstrates that the inductance characteristic depends on the high magnetic permeability material used and the thickness of the material, where 0.014, 0.006 and 0.020 inches represent the thickness of the cores. As compared to the two supermalloys, PERMALLOY™ 0.020 provides a steeper rate of change between inductance and needle extension; it may therefore provide better resolution of the particular needle extension given a particular inductance measurement. Therefore, the choice of a particular material and the thickness of that material can be used to create a core having the desired rate of change in inductance relative to needle extension.

In some embodiments, multiple high magnetic permeability cores 80 are mounted to a deployable needle, as shown in FIG. 3. The inductance characteristics may be similarly shaped to the curves shown in FIG. 4 in such embodiments if the associated receiver coil 50 is large in comparison to the high magnetic permeability cores 80 or if the high magnetic permeability cores are closely spaced together. In some instances, where the high magnetic permeability cores 80 are spaced apart or the receiver coil 50 is sized similar to the high magnetic permeability cores 80, the inductance characteristic will show a series of peaks associated with the extension distance where each high magnetic permeability core 80 passes directly beneath the receiver coil 50. In such embodiments, the extension analysis system 70 gauges the peak to peak changes in inductance to determine the amount of extension of the deployable object 40.

The extension analysis system provides an algorithm which receives the induction or the induced current data and produces data regarding extension of the deployable object 40. In some embodiments, the extension analysis system is a part of the navigation system. In other embodiments, the extension analysis system is provided separate from the navigation system. In some embodiments, the data regarding extension of the deployable object 40 may be provided to the operator in the form of a visual display. Such display may be continuously provided throughout the procedure or may be provided only upon demand by the operator. For example, in one embodiment, the extension analysis system is included in the navigation system. The operator may use the navigation system to position the medical instrument and may then switch the system to receiving and providing data regarding extension of the deployable object 40. Once the extension of the deployable object 40 is complete, the operator may then switch the system back to operating as a navigation system.

In some embodiments of the present disclosure, the use of an electromagnetic source may be eliminated. In such embodiments, the inductance may be measured directly via sending a signal across the receiver coil 50 and measuring the response. That is, the inductance, and therefore the position of the deployable object, is measured electrically by the extension analysis system 70 without the induction of a current into the receiver coil 50 by an electromagnetic source.

Embodiments of the present disclosure may be used in combination with electromagnetic navigation systems such as the navigation system disclosed in U.S. patent application Publication Number 2007/0164900, the relevant portions of which are hereby incorporated by reference. Certain devices are designed to use an electromagnetic source and electromagnetic navigation receiver coils 90 (see FIG. 2) for minimally invasive surgical implantation procedures. Like the extension receiver coils 50, the current of each navigation receiver coil 90 is dependent upon the location and orientation of the respective navigation receiver coil 90 within the magnetic field. The navigation coils 90 are connected to conductors 95 which conduct the current to a navigation analysis system 100. By sensing and processing current conducted from each navigation receiver coil 90, a navigation analysis system 100 can determine the location of each navigation receiver coil with respect to one another and provide a visual map to aid the operator in navigating the device to a target site within the body of the patient. Such systems provide the advantages of imaging with reduced radiation exposure and provide three dimensional imaging. An example of a navigation analysis system is the system used in the Medtronic Stealth Station. The navigation analysis system and the extension analysis systems may comprise separate systems or may be incorporated together into a single system. In some embodiments, one or more electromagnetic emitters are attached to a fluoroscopy unit, such as to the C-arm of the unit. For example, one or more emitters 110 may be located about 10 to 15 centimeters from the patient and may emit at a low power, such as one-half Watt. By emitting at a low power, the emitters 110 create less noise in the operative field and therefore cause less interference. Alternatively, one or more emitters 110 may be located underneath the fluoroscopy table. In other embodiments, a very small emitter coil may be located in the medical instrument, proximal to the receiver coil by about 10-30 millimeters, for example.

Emitters 110 may also be employed for use with the extension analysis system 70, receiver coil 50, and deployable object 40 with high magnetic permeability cores 80. In some embodiments of the present disclosure, the medical instrument includes one or more extension electromagnetic receiver coils 50 for detection of extension of the deployable object 40 and one or more separate navigation electromagnetic receiver coils 90 for navigation. In such embodiments, the two types of electromagnetic receiver coils need not be the same size. For example, the one or more extension electromagnetic receiver coils 50 for detection of extension of the deployable object 40 may be smaller and therefore closer to the deployable object 40 than the navigation coils 90. An example of such an embodiment is shown in FIG. 2. In some embodiments employing both extension receiver coils 50 and navigation receiver coils 90, a first electromagnetic emitter 110 emits an electromagnetic signal at a first frequency tuned to the navigation receiver coil or coils 90 and a second electromagnetic emitter 110 emits an electromagnetic signal at a second frequency, different from the first frequency, tuned to the extension receiver coils 50 which determine the amount of needle extension. In this way, the system may avoid potential interference between the navigation and the extension detection functions. Alternatively, instead of using separate frequencies, the emitters 110 may emit electromagnetic signals of the same frequency but at different times, such as in rapid succession to one another in a time division multiplexing scheme. Potential changes in inductance could be characterized and compensated for when a high permeability core 50 is within proximity of a navigation receiver coil 90, such as within 2 or 3 millimeters.

In some embodiments of the present disclosure, one or more electromagnetic receiver coils are used for detection of both extension of the deployable object and for navigation. In such embodiments, the electromagnetic receiver coil may be connected to both the extension analysis system 70 and the navigation analysis system 100 or the connection may be switched between them. During navigation activities, the receiver coil functions with the navigation analysis system, and during extension analysis, the receiver coil functions with the extension analysis system 70.

In embodiments including navigation, the electromagnetic receiver coils for navigation may be used to provide the location of the medical instrument within the patient. One navigation system useful with embodiments of the present disclosure is a system often called virtual fluoroscopy. In virtual fluoroscopy, an analysis component of the navigation system 100 processes current signals from the navigation electromagnetic receiver assemblies 90 to create a virtual image of the medical instrument. Embodiments may use the known extension of the deployable object 40 as determined by inductance to also create a virtual image of the deployable object 40 as it extends from the virtual image of the distal tip of the medical instrument.

In some embodiments of the present disclosure, the extension analysis system 70 may also include a control mechanism for effecting a particular extension of the deployable object. In such embodiments, the extension analysis system 70 includes a motor or a control signal line to a motor that moves the deployable object relative to the medical instrument. The motor may be of any appropriate type, such as a DC stepper motor. The extension analysis system 70 may also include a user interface where the user may set a desired extension distance. The extension analysis system, when enabled, will then measure the extension and control the motor to effect the proper extension. Alternatively, in some embodiments, the one or more extension electromagnetic receiver coils 50 may be located on the deployable object 40 and the one or more high magnetic permeability cores 80 may be located in the medical instrument. Such embodiments would function to measure extension of the deployable object 40 by measuring induction as the receiver coils 50 on the deployable object move relative to the stationary high magnetic permeability cores 80.

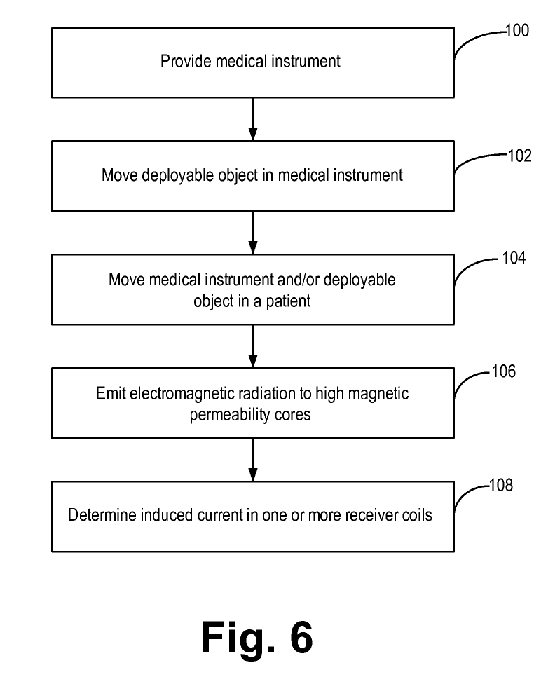

FIG. 6 is a flow diagram that depicts a method of using a medical instrument. At operation 100, a medical instrument is provided. The medical instrument can include one or more electromagnetic receiver coils. The one or more receiver coils can be electrically connectable to an extension analysis system. At operation 102, a deployable object can move within the medical instrument. The deployable object can include one or more high magnetic permeability cores. The high magnetic permeability cores can be located proximate to the one or more receiver coils. At operation 104, the medical instrument and/or the deployable object can be moved within a patient. At operation 106, electromagnetic radiation can be emitted to the high magnetic permeability cores. At operation 108, the induced current in one or more receiver coils can be determined. Determining induced current can be performed by measuring or predicting the value of the amperes of the induced current. Predicting the induced current can be performed through employing statistical methods along with sensing of values associated with certain electrical parameters such as current or voltage across two terminals.

The description of the present invention is merely exemplary in nature and, thus, variations that do not depart from the gist of the invention are intended to be within the scope of the invention. Such variations are not to be regarded as a departure from the spirit and scope of the invention.

What is claimed is:

1. An inductive measurement system for a medical instrument comprising:
a receiver coil mounted proximate to a distal end of the medical instrument, the receiver coil being electrically connectable to an extension analysis system at a proximal end of the medical instrument;
a deployable object disposed and movable within the medical instrument, the deployable object bearing a high magnetic permeability material located proximate to the receiver coil, the high magnetic permeability material and the receiver coil combining to form an inductive element having an inductance that varies in a predetermined manner with the position of the deployable device relative to the receiver coil; and
an electromagnetic emitter for emitting an electromagnetic wave that induces a current in the receiver coil, the inductance of the inductive element being measurable by the extension analysis system based on the induced current, whereby the extension of the deployable device through the catheter can be determined.

2. The system of claim 1, wherein the medical instrument being a catheter and the deployable object being a needle.

3. The system of claim 2, wherein the high magnetic permeability material includes one or more ferrous cores.

4. The system of claim 3, wherein the one or more ferrous cores being located proximate a distal tip of the needle.

5. The system of claim 2, wherein the receiver coil forms a loop around a lumen of the delivery catheter.

6. The system of claim 1, wherein the medical instrument being a chronic total occlusion catheter and the deployable object being a chronic total occlusion crossing guidewire.

7. The system of claim 1, wherein the deployable object includes three ferrous cores.

8. A medical instrument assembly comprising:
a medical instrument having proximal and distal ends;
a receiver coil mounted proximate to a distal end of the medical instrument, the receiver coil being electrically connectable at a proximal end of the medical instrument;
a deployable object disposed and movable within the medical instrument, the deployable object bearing a high magnetic permeability material located proximate to the receiver coil, the high magnetic permeability material and the receiver coil combining to form an inductive element having an inductance that varies in a predetermined manner with the position of the deployable device relative to the receiver coil, whereby a determination of the inductance at the proximal end of the delivery catheter indicates the extension of the deployable device through the medical instrument.

9. The system of claim 8, wherein the medical instrument being a catheter and the deployable object being a needle.

10. The system of claim 9, wherein the high magnetic permeability material being located near a distal tip of the needle.

11. The system of claim 9, wherein the high magnetic permeability material comprises a ferrous core.

12. The system of claim 8, wherein the receiver coil being a navigation coil.

13. The system of claim 8, further including an electromagnetic emitter for inducing a current in the receiver coil, the induced current being used to determine the inductance of the inductive element.

14. The system of claim 8, wherein the electromagnetic emitter emits electromagnetic radiation to determine the inductance of the inductive element and to provide navigation information for the delivery catheter.

15. A method of assessing the extension of a deployable object from a medical instrument comprising:
providing a medical instrument having one or more electromagnetic receiver coils, the one or more receiver coils being electrically connectable to an extension analysis system;
providing a deployable object movable within the medical instrument, the deployable object including one or more high magnetic permeability cores, the high magnetic permeability cores being located proximate to the one or more receiver coils;
inserting the medical instrument and the deployable object within a patient;
extending the deployable object from a distal end of the medical instrument;
emitting electromagnetic radiation proximate to the high magnetic permeability cores and one or more receiver coils to induce a current in the one or more receiver coils;
determining the induced current to determine the extension of the deployable object through the medical instrument.

16. The method of claim 15, wherein the medical instrument being a catheter and the deployable object being a needle.

17. The method of claim 16, wherein the one or more high magnetic permeability cores being near the distal tip of the needle.

18. The method of claim 16, wherein the one or more high magnetic permeability cores comprise one or more ferrous cores.

19. The method of claim 16, wherein the one or more electromagnetic receiver coils form a loop around a lumen of the catheter.

20. The method of claim 15, wherein the extension analysis system includes a control feedback loop.

21. A method of using a medical instrument comprising:
providing the medical instrument having one or more electromagnetic receiver coils, the one or more receiver coils being electrically connectable to an extension analysis system;
moving a deployable object within the medical instrument, the deployable object including one or more high magnetic permeability cores, the high magnetic permeability cores being located proximate to the one or more receiver coils;
moving one of the medical instrument and the deployable object within a patient;
emitting electromagnetic radiation to the high magnetic permeability cores; and
determining the induced current.
